(19) United States
(12) Patent Application Publication
(10) Pub. No.: US 2009/0081318 A1
(43) Pub. Date: Mar. 26, 2009

(54) TREATMENT OF NEUROADS USING INHIBITORS OF GLYCOGEN SYNTHASE KINASE (GSK)-3

(76) Inventors: Harris A. Gelbard, Pittsford, NY (US); Sanjay B. Maggirwar, Rochester, NY (US); Stephen Dewhurst, Rochester, NY (US); Giovanni Schifitto, Rochester, NY (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/158,896

(22) PCT Filed: Dec. 19, 2006
(86) PCT No.: PCT/US2006/062329

§ 371 Date: Oct. 29, 2008

Related U.S. Application Data
(60) Provisional application No. 60/753,614, filed on Dec. 23, 2005.

(51) Int. Cl. A61K 33/00; A61K 31/55; A61K 31/19

(52) U.S. Cl. 424/722; 514/215; 514/557

(57) ABSTRACT

Provided is a method of treating or preventing neurological disease in a subject in need of such treatment or prevention, comprising administering to the subject a therapeutically effective dose of a GSK-3 inhibitor.

### CROSS-REFERENCE TO RELATED APPLICATIONS

This application claims benefit of U.S. Provisional Application No. 60/753,614, filed Dec. 23, 2005, which is hereby incorporated herein by reference in its entirety.

### STATEMENT REGARDING FEDERALLY SPONSORED RESEARCH

This invention was made with government support under Grants PO1 MH64570, T32 AI49815, PO1 AI050244 and 5T32DA007232 awarded by the National Institutes of Health, Grant PO1MH64570 awarded by the National Institute of Mental Health, and Grant ES07026 awarded by the NIEHS.

### BACKGROUND OF THE INVENTION

Cognitive impairment continues to be a frequent co-morbidity associated with HIV infection despite the use of highly active antiretroviral therapy (HAART)(Sacktor N, et al. 2002), underscoring the need for adjunctive therapies. HIV-1 does not induce disease by direct infection of neurons, although extensive data suggest that intra-CNS viral burden correlates with both the severity of virally-induced neurologic disease, and with the generation of neurotoxic metabolites. Many of these molecules are capable of inducing neuronal apoptosis in vitro, but neuronal apoptosis in vivo does not correlate with CNS dysfunction. Thus, the mechanism of virally-induced neurologic disease is not known in the literature. HIV-1 neurotoxins including platelet activating factor (PAF) and Tat activate glycogen synthase kinase (GSK)-3β. Disclosed herein are methods of treating neurological disease using GSK-3 inhibitors.

### BRIEF SUMMARY OF THE INVENTION

Provided is a method of treating or preventing neurological disease in a subject in need of such treatment or prevention, comprising administering to the subject a therapeutically effective dose of a GSK-3 inhibitor.

Also provided is a method of treating or preventing HIV-1 associated dementia (HAD) in a subject in need of such treatment or prevention, comprising administering to the subject a therapeutically effective dose of Valproic acid.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION OF THE INVENTION

The disclosed methods and compositions may be understood more readily by reference to the following detailed description of particular embodiments and the Example included therein and to the Figures and their previous and following description.

Provided are methods for treating neurological disorders by administering a GSK-3β inhibitor. Thus, disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a GSK-3β inhibitor is disclosed and discussed and a number of modifications that can be made to the GSK-3β inhibitor are discussed, then each and every combination and permutation of the GSK-3β inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Provided herein is a method of treating or preventing neurological disease in a subject in need of such treatment or prevention, comprising administering to the subject a therapeutically effective dose of a GSK-3 inhibitor. The neurological disease of the provided method can be HIV-1 associated dementia (HAD). Thus, the method can further comprise the step of diagnosing the subject with HAD. HAD is comprised of a spectrum of conditions from the mild HIV-1 minor cognitive-motor disorder (MCMD) to severe and debilitating AIDS dementia complex. Symptoms begin with motor slowing and may progress to severe loss of cognitive function, loss of bladder and bowel control, and paraparesis. A classification system has been formulated for HIV associated dementia, wherein subjects are classified as being Stage 0 (Normal), Stage 0.5 (Subclinical or Equivocal), Stage 1 (Mild), Stage 2 (Moderate), Stage 3 (Severe), or Stage 4 (End-Stage). Thus, the subject of the provided method can therefore be classified as Stage 0, Stage 0.5, Stage 1, Stage 2, Stage 3, or Stage 4.

By “treat” or “treatment” is meant a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for treatment of HAD is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. For example, in the case of HAD, to treat HAD in a subject can comprise improving the disease classification. (e.g. from stage 3 to stage 2, from stage 2 to stage 1, from stage 1 to 0.5 or from stage 0.5 to 0).

As used throughout, “prevent” means to preclude, avert, obviate, forestall, stop, or hinder something from happening, especially by advance planning or action. For example, to prevent HAD in a subject is to stop or hinder the subject from advancing in disease classification (e.g. from stage 0 to stage 0.5, from stage 0.5 to stage 1, from stage 1 to stage 2, from stage 2 to stage 3, or from stage 3 to stage 4).

GSK-3 is a protein kinase found in a variety of organisms, including mammals. Two nearly identical forms of GSK-3 exist: GSK-3α and GSK-3β. The inhibitor can be any known or newly discovered GSK-3 inhibitor. Optimally, the GSK-3 inhibitor of the provided method inhibits at least GSK-3β. The amino acid sequence for human GSK-3β can be accessed at Genbank accession number P49841, and the corresponding nucleotide sequence at accession number NM-002093. For experimental and screening purposes, it may be desirable to use an animal model. For example, the rat GSK-30 sequence may be accessed at Genbank accession number P18266, and the mouse at Genbank accession number AAD39258.

GSK-3 inhibitors, as used herein, are compounds that directly or indirectly reduce the level of GSK-3 activity in a cell, by competitive or non-competitive enzyme inhibition; by decreasing protein levels, e.g. by a targeted genetic disruption, reducing transcription of the GSK-3 gene, increasing protein instability, etc. Inhibitors may be small organic or inorganic molecules, anti-sense nucleic acids, antibodies or fragments derived therefrom, etc. Other inhibitors of GSK-3 can be found through screening combinatorial or other chemical libraries for the inhibition of GSK-3 activity.

Optimally, the GSK-3 inhibitor of the provided method is valproic acid (VPA) or an analog, derivative, or pharmaceutically acceptable salt of VPA. U.S. patent application Ser. No. 09/929,810 (Nau et al) and U.S. patent application Ser. No. 09/840,376 (Nau et al) are incorporated by reference herein in their entirety for their teaching of valproic acid analogs and derivatives. Thus, also provided is a method of treating or preventing HIV-1 associated dementia (HAD) in a subject in need of such treatment or prevention, comprising administering to the subject a therapeutically effective dose of Valproic acid, or an analog, derivative, or pharmaceutically acceptable salt thereof. Valproic acid (VPA) is a potent broad-spectrum anti-epileptic with demonstrated efficacy in the treatment of bipolar affective disorder. VPA inhibits both GSK-3α and GSK-3β, with significant effects observed at concentrations of VPA similar to those attained clinically (Chen et al. 1999).

For example, the GSK-3 inhibitor of the provided method can be a compound having a structure represented by the formula:

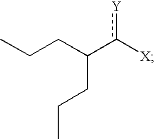

wherein “—” is a single or a double covalent bond;
wherein X is OH, SH, NH₂, NHR, NR₂, O⁻ Z⁺, or absent, wherein each R is independently selected from alkyl, alkenyl, alkynyl, aryl, acyl, and carbonyl, and
wherein Z is a cation;
wherein Y is O, S, N, or NH; and
wherein the structure can be further substituted.

In some aspects, the cation is a monovalent cation selected from lithium, sodium, and potassium. In some aspects, X is OH and Y is O. In some aspects, “—” is a double covalent bond, Y is N, and X is absent. In some aspects, Y is O, X is O⁻ Z⁺, and Z is lithium or sodium.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 20 carbon atoms, for example 1 to 10 or 1 to 6 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkenyl” as used herein is a hydrocarbon group of from 2 to 40 carbon atoms, for example from 2 to 20 or from 2 to 10 carbon atoms, with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 40 carbon atoms, for example from 2 to 20 or from 2 to 10 carbon atoms, with a structural formula containing at least one carbon-carbon triple bond. The alkynyl group can be unsubstituted or substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “amine” or “amino” as used herein are represented by the formula NA¹A²A³, where A¹, A², and A³ can be, independently, hydrogen or substituted or unsubstituted alkyl, cycloalkyl, alkenyl, cycloalkenyl, alkynyl, cycloalkynyl, aryl, or heteroaryl group as described herein.

The term “carboxylic acid” as used herein is represented by the formula —C(O)OH.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, a substituted or unsubstituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “hydroxyl” as used herein is represented by the formula —OH.

The term “ketone” as used herein is represented by the formula A¹C(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Examples of direct inhibitors of GSK-3 protein include lithium (Li⁺) (Klein et al. 1996), which potently inhibits GSK-3β activity (Kᵢ=2 mM), but is not a general inhibitor of other protein kinases. Beryllium ions (Be²⁺) are stronger inhibitors of GSK-3, inhibiting in the micromolar range. However, this inhibitory effect is not as selective as lithium because it will also inhibit CDK1 at low doses.

TABLE 1
Inhibitors of GSK-3.

| | Inhibition | Interaction | |
|---|---|---|---|
| Inhibitor | potency | type | Notes: |
| Bisindole maleimides | IC₅₀ = 5-170 nM | ATP | Also potent inhibitors of |
| (e.g. Ro 31-8220, GF 109203x) | | competitor | PKC |
| Anilino maleimides | Ki = 10-30 nM | ATP | Inactive on a range of |
| (e.g. SB-216763 & SB-415286) | | competitor | other kinases |
| Aldisine alkaloids | IC₅₀0 = 10 nM | ATP | Also potent inhibitors of |
| (hymenialdisine) | | competitor | MEK's, CK1 and CDK's |
| Paullones | IC₅₀ = 4-80 nM | ATP | Also inhibitors of CDK's |
| (e.g. alsterpaullone) | | competitor | and mMDH |
| Indirubins | IC₅₀ = 5-50 nM | ATP | Also potent inhibitors of |
| (e.g. indirubin-3′-monoxime) | | competitor | CDK's |
| Pyrazoloquinoxalines | IC₅₀ = 1 μM | ATP | Also potent inhibitors of |
| (e.g. 3-amino-2-quinoxaline | | competitor | CDK's |
| carbonitrile) | | | (IC₅₀ = 500 nM) |
| Thiadiazolidinones | IC₅₀ = 2 μM | Unknown | Inactive to 100 uM on |
| (e.g. 4-benzyl-2-methyl-1,2,4- | | | CDK1/cyclin B, CK2, |
| thiadiazolidine-3,5-dione) | | | PKA and PKC |
| Lithium | Ki = 2 mM | Mg | Also IMPase inhibitor |
| | | competitor | |
| Beryllium | IC₅₀ = 6 μM | Mg and | Inhibitor of CDK1 |
| | | ATP | (IC₅₀ = 50 uM) |
| | | competitor | |
| Pseudosubstrate peptide | Ki = 0.7 mM | Substrate | Specific |
| (GRPRTTS*FAE; SEQ ID NO: 1) | | competitor | |

CDK = Cyclin-Dependent Kinase,
MEK-1 = mitogen activated protein/ERK kinase 1,
mMDH = Mitochondrial Malate Dehydrogenase,
IMPase = Inositot monophosphatase,
CK = Casein Kinase,
PKC = Protein Kinase C,
PKA = Protein Kinase A,
S* = Phosphoserine.

A number of other compounds have been found to inhibit GSK-3 (Table 1). The majority inhibit kinase activity through interaction with the ATP-binding site. They include Bisindole- and Anilino maleimides, Aldisine alkaloids, Paullones, Indirubins and Pyraloquinoxalines. For example, Paullones and their use in GSK-3 inhibition is described, for example, in Kunick C, et al. J Med. Chem. 2004 Jan. 1; 47(1):22-36, which is hereby incorporated by reference herein in its entirety for its teaching of Paullones. Such compounds are effective at nanomolar concentrations in vitro and low micromolar in vivo. Again, whilst many have been shown to be potent, they are not very specific to GSK-3 and commonly inhibit the related CDKs at similar levels. However, two structurally distinct maleimides (SB216763 and SB415286) have been shown to be potent and to have high specificity for GSK-3. They can effectively substitute for lithium as GSK-3 inhibitors in cell studies. Members of the class of compounds termed granulatimides or didemnimides have also been found to act as GSK-3 inhibitors (International patent application WO 99/47522, which is hereby incorporated herein for its teaching of these compounds).

Some indirect inhibitors of GSK-3 include wortmannin, which activates protein kinase B, resulting in the phosphorylation and inhibition of GSK-3. Isoproterenol, acting primarily through beta3-adrenoreceptors, decreases GSK-3 activity to a similar extent (approximately 50%) as insulin (Moule et al. 1997). p70 S6 kinase and p90rsk-1 also phosphorylate GSK-3β, resulting in its inhibition.

GSK-3 can also be selectively targeted using GSK-3-specific peptides. For example, frequently rearranged in advanced T-cell lymphomas 1 (FRAT1) is a mammalian homologue of a GSK3-binding protein (GBP). FRATtide (a peptide corresponding to residues 188-226 of FRAT1) binds to GSK3 and blocks the GSK3-catalysed phosphorylation of Axin and beta-catenin (Thomas G M, et al. FEBS Lett. 1999 Sep. 17; 458(2):247-51).

The GSK-3 inhibitor of the provided method can also be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of GSK-3 or the genomic DNA of GSK-3 or they can interact with the polypeptide GSK-3. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10-6, 10-8, 10-10, or 10-12. A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10-12 M. It is preferred that the aptamers bind the target molecule with a K_{d} less than 10-6, 10-8, 10-10, or 10-12. Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III—like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for c-Kit or SCF. For example, siRNAs for silencing gene expression of c-Kit is commercially available (SURESILENCING™ Human c-Kit siRNA; Zymed Laboratories, San Francisco, Calif.).

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

Antibodies can also be used to directly inhibit GSK-3 protein. Antibodies may be prepared in accordance with conventional ways, where the GSK-3 or a fragment thereof is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

The provided method can further comprise administering to the subject other compositions known or newly discovered to be beneficial in the treatment of neurological disease. For example, the provided method can further comprise administering to the subject a therapeutically effective dose of an inhibitor of mitochondrial hyperpolarization (MHP). Specific examples of inhibitors of MHP and their efficacy in treating HAD are disclosed in U.S. Application No. 60/663,424 (Perry et al), which is hereby incorporated by reference in its entirety at least for its teaching and exemplification of inhibition of MHP.

As used herein, mitochondrial hyperpolarization (MHP) refers to an elevation in the mitochondrial transmembrane potential, ΔΨₘ (delta psi), i.e., negative inside and positive outside). The ΔΨₘ is the result of an electrochemical gradient maintained by two transport systems—the electron transport chain and the F₀F₁-ATPase complex. For a review, see Perl et al. 2004 Trends in Immunol. 25:360-367. Briefly, the electron transport chain catalyzes the flow of electrons from NADH to molecular oxygen and the translocation of protons across the inner mitochondrial membrane, thus creating a voltage gradient with negative charges inside the mitochondrial matrix. F₀F₁-ATPase utilizes the extruded proton to synthesize ATP. MHP leads to uncoupling of oxidative phosphorylation, which disrupts ΔΨₘ and damages integrity of the inner mitochondrial membrane. Disruption of ΔΨₘ has been proposed as the point of no return in cell death signaling. This releases cytochrome c and other cell-death-inducing factors from mitochondria into the cytosol. Thus, the inhibitor of MHP can be a F₀F₁-ATPase agonists.

KATP channels participate in controlling plasma and mitochondrial membrane polarity, by controlling K⁺ efflux at the plasma membrane, and K⁺/H⁺ exchange at the mitochondrial membrane. As such, both plasma membrane and mitochondrial membrane KATP channels can effect mitochondrial polarization. Thus, the inhibitor of MHP can be a KATP channel antagonist. The KATP channel antagonist can be selected from the group consisting of Tolbutamide, hydroxydecanoic acid (5-HD), glibenclamide (glyburide), and meglitinide analog (e.g. Repaglinide, A-4166).

The inhibitor of MHP can be an electron transport inhibitor. The electron transport chain (ETC) is the biomolecular machinery present in mitochondria that couples the flow of electrons to proton pumps in order to convert energy from sugar to ATP. The electron transport chain couples the transfer of an electron from NADH (nicotinamide adenine dinucleotide) to molecular oxygen (O₂) with the pumping of protons (H⁺) across a membrane. The charge gradient that results across the membrane serves as a battery to drive ATP Synthase. The electron transport chain is made up of several integral membrane complexes: NADH dehydrogenase (complex I), Coenzyme Q—cytochrome c reductase (complex III), and Cytochrome c oxidase (complex IV). Succinatie—Coenzyme Q reductase (Complex II) connects the Krebs cycle directly to the electron transport chain.

Thus, the inhibitor of MHP can be an inhibitor of any component of the ETC. Thus, the inhibitor can be an inhibitor of complex I, II, III, or IV. For example, diphenylene iodonium (DPI) and rotenone are specific inhibitors of complex I, succinate-q reductase (TTFA) is an inhibitor of complex II, antimycin A and myxothiazole are inhibitors of complex III, and potassium cyanide (KCN) is an inhibitor of complex IV. Thus, the inhibitor of MHP can be selected from the group consisting of diphenylene iodonium (DPI), rotenone, antimycin, myxothiazole, succinate-q reductase (TTFA), and potassium cyanide (KCN).

The inhibitor of MHP can be an uncoupler. As used herein an “uncoupler” is a substance that allows oxidation in mitochondria to proceed without the usual concomitant phosphorylation to produce ATP; these substances thus “uncouple” oxidation and phosphorylation. As an example, Trifluorocarbonylcyanide Phenylhydrazone (FCCP) is a chemical uncoupler of electron transport and oxidative phosphorylation. FCCP permeabilizes the inner mitochondrial membrane to protons, destroying the proton gradient and, in doing so, uncouples the electron transport system from the oxidative phosphorylation system. In this situation, electrons continue to pass through the electron transport system and reduce oxygen to water, but ATP is not synthesized in the process. The uncoupler of the present method can agonize, antagonize or modulate the expression of endogenous mitochondrial uncoupling proteins (UCPs). As a non-limiting example, the uncoupler of the present method can be the beta-adrenergic agonist CL-316,243 (disodium (R,R)-5-(2-((2-(3-chlorophenyl)-2-hydroxyethyl)-amino)propyl)-1,3-benzodioxole-2,3-dicarboxylate) (Yoshida et. al., Am J Physiol. 1998. 274(3 Pt 1): p. E469-75). The uncoupler of the present method can be a protonophore. Thus, the inhibitor of MHP can be a protonophore. As used herein, a “protonophore” is a molecule that allows protons to cross lipid bilayers. The protonophore can be FCCP. The protonophore can also be 2,4,-dinitrophenol (DNP). The protonophore can be also m-chlorophenylhydrazone (CCCP). The protonophore can also be pentachlorophenol (PCP).

The disclosed method can further comprise administering to the subject a therapeutically effective dose of a modulator of adenosine receptor signaling. Specific examples modulator of adenosine receptor signaling and their efficacy in treating HAD are disclosed in U.S. Application No. 60/663,059 (Dewhurst et al), which is hereby incorporated by reference in its entirety at least for its teaching and exemplification of modulating adenosine receptor signaling.

Endogenous adenosine plays a pivotal role in the regulation of neural cell fate. The actions of adenosine are mediated by specific receptors located on cell membranes, which belong to the family of G protein-coupled receptors. Currently, four adenosine receptors have been cloned: A₁, A_{2A}, A_{2B}, and A₃. The disclosed modulator of adenosine receptor signaling can comprise any composition that will alter a biological property of either adenosine or adenosine receptors in a cell, such as for example their synthesis, degredation, translocation, binding, or phosphorylation, such that the alteration results in a net increase or decrease in adenosine receptor signaling in the cell. As a non-limiting example, the provided modulator can be a nucleic acid that alters expression of either adenosine or adenosine receptor in a cell, such as for example RNAi or antisense nucleic acids. As another example, the provided modulator can be a polypeptide that alters the binding of adenosine to adenosine receptors, such as for example soluble adenosine receptors, mutant adenosine ligands or antibodies specific for adenosine or adenosine receptors. As another example, the provided modulator can comprise informational molecules that modulate adenosine receptor expression (such as short-interfering RNAs or peptide nucleic acids) or molecules that may regulate downstream signaling events that may occur as a result of adenosine receptor stimulation.

Thus, the provided modulator of adenosine receptor signaling can be a small molecule comprising a modified adenosine (6-amino-9-beta-D-ribofuranosyl-9-H-purine). Modifications that can be made to adenosine are well known in the art. These modifications include those that result in adenosine receptor agonists and antagonists. These agonists and antagonists can be either receptor selective or non-selective. Provided herein is the use of these adenosine receptor agonists and antagonists in the treatment of HAD.

The modulator of the present method can be an adenosine 1 receptor (A₁R) antagonist. The modulator can be an adenosine 2A receptor (A_{2A}R) antagonist. The modulator can be an adenosine 2B receptor (A_{2B}R) antagonist. The modulator can be an adenosine 3 receptor (A₃R) antagonist. Thus, the modulator can be any adenosine receptor selective antagonist, whether known in the art or later developed. Non-limiting examples of A_{2A}R selective antagonists include ATL455, ZM241385, KW-6002 (istradefylline), SCH 58261, and the pharmaceutically acceptable salts thereof. ZM241385 is 4(2-[7-Amino-2-(2-furyl)[1,2,4]triazolo[2,3-a][1,3,5]triazin-5-ylamino]ethyl)phenol (Poucher et al. 1995; Poucher et al 1996; Keddie et al 1996). KW-6002 (istradefylline) is (E)-1,3-diethyl-8-(3,4-dimethoxystyryl)-7-methyl-3,7-dhydro-1H-purine-2,6-dione. KW-6002 has been evaluated humans as a treatment for Parkinson's disease (Bara-Jimenez et al. 2003). SCH 58261 is 7-(2-phenylethyl)-5-amino-2-(2-furyl)-pyrazolo-[4,3-e]-1,2,4-triazolo[1,5-c]pyrimidine.

These modifications to adenosine to produce antagonists are exemplary and provide guidance to and description for other antagonistic adenosine modifications.

The provided modulator can be an adenosine 1 receptor (A₁R) agonist. The modulator can be an adenosine 2A receptor (A_{2A}R) agonist. The modulator can be an adenosine 2B receptor (A_{2B}R) agonist. The modulator can be an adenosine 3 receptor (A₃R) agonist, such as for example CF101 (Aderis Pharmaceuticals, Hopkinton, Mass.). Thus, the provided modulator can be any adenosine receptor selective agonist, whether known in the art or later developed. Non-limiting examples of A_{2A}R selective agonist include ATL146e, ATL313, PJ-1165, Binodenoson (MRE-0470), MRE-0094, CGS21680, and the pharmaceutically acceptable salts thereof. ATL146e is 4-{3-[6-amino-9-(5-ethylcarbamoyl-3,4-dihydroxytetrahydrofuran-2-yl)-9H-purin-2-yl]prop-2-ynyl}cyclohexanecarboxylic acid methyl ester (Lappas C M, et al. 2005). ATL313 is 4-{3-[6-amino-9-(5-cyclopropylcarbamoyl-3,4-dihydroxytetrahydrofuran-2-yl)-9H-purin-2-yl]prop-2-ynyl}piperidine-1-carboxylic acid methyl ester (Lappas C M, et al. 2005). CGS21680 is 4-[2-[[6-Amino-9-(N-ethyl-b-D-ribofuranuronamidosyl)-9H-purin-2-yl]amino]ethyl]benzenepropanoic acid hydrochloride (Phillis et al 1990; Nekooeian and Tabrizchi 1998; Klotz 2000). These modifications to adenosine to produce agonists are exemplary and provide guidance to and description for other agonistic adenosine modifications.

The disclosed method can further comprise administering to the subject a therapeutically effective dose of an antioxidant. Generally, antioxidants are compounds that react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The herein disclosed antioxidant can be any antioxidant, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly α-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants.

Thus, the disclosed method can further comprise administering to the subject a therapeutically effective dose of an antioxidant selected from the group consisting of tauroursodeoxycholic acid (TUDCA), N-acetylcysteine (NAC) (600-800 mg/day), Mito-Coenzyme Q10 (Mito-CoQ) (300-400 mg/day), Mito-VitaminE (Mito-E) (100-1000 mg/day), Coenzyme Q10 (300-400 mg/day), and idebenone (60-120 mg/day).

The disclosed method can further comprise administering to the subject a therapeutically effective dose of an antiretroviral compound. Antiretroviral drugs inhibit the reproduction of retroviruses such as HIV. Antiretroviral agents are virustatic agents which block steps in the replication of the virus. The drugs are not curative; however continued use of drugs, particularly in multi-drug regimens, can significantly slow disease progression. There are three main types of antiretroviral drugs, although only two steps in the viral replication process are blocked. Nucleoside analogs, or nucleoside reverse transcriptase inhibitors (NRTIs), act by inhibiting the enzyme reverse transcriptase. Because a retrovirus is composed of RNA, the virus must make a DNA strand in order to replicate itself. Reverse transcriptase is an enzyme that is essential to making the DNA copy. The nucleoside reverse transcriptase inhibitors are incorporated into the DNA strand. This is a faulty DNA molecule that is incapable of reproducing. The non-nucleoside reverse transcriptase inhibitors (NNRTIs) act by binding directly to the reverse transcriptase molecule, inhibiting its activity. Protease inhibitors act on the enzyme protease, which is essential for the virus to break down the proteins in infected cells. Without this essential step, the virus produces immature copies of itself, which are non-infectious. A fourth class of drugs called fusion inhibitors block HIV from fusing with healthy cells.

Thus, the antiretroviral compound can comprise one or more molecules selected from the group consisting of protease inhibitors (PI), fusion inhibitors, nucleoside reverse transcriptase inhibitors (NRTI), and non-nucleoside reverse transcriptase inhibitors (NNRTI). The antiretroviral compound of the provided method can be a PI, such as a PI selected from the group consisting of Indinavir, Amprenavir, Nelfinavir, Saquinavir, Fosamprenavir, Lopinavir, Ritonavir, and Atazanavir, or any combinations thereof. The antiretroviral compound of the provided method can be a fusion inhibitor, such as for example Enfuvirtide. The antiretroviral compound of the provided method can be a NRTI, such as a NRTI selected from the group consisting of Abacavir, Stavudine, Didanosine, Lamivudine, Zidovudine, Zalcitabine, Tenofovir, and Emtricitabine, or any combinations thereof. The antiretroviral compound of the provided method can be a NNRTI, such as a NNRTI selected from the group consisting of Efavirenz, Nevirapine, and Delavirdine.

The disclosed method can further comprise administering to the subject a neurotoxin inhibitor. The inhibitor can be a TNFα inhibitor, including TNFα-inhibitory monoclonal antibodies (e.g., etanercept), phosphodiesterase (PDE)-4 inhibitors (such as IC485, which can reduce TNFα production), thalidomide and other agents. Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the CH2 domain, the CH3 domain and hinge region, but not the CH1 domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept has been evaluated in HIV-infected subjects receiving highly active antiretroviral therapy (HAART) (Sha B E, Valdez H, Gelman R S, Landay A L, Agosti J, Mitsuyasu R, Pollard R B, Mildvan D, Namkung A, Ogata-Arakaki D M, Pox L, Estep S, Erice A, Kilgo P, Walker R E, Bancroft L, Lederman M M. Effect of etanercept (Enbrel) on interleukin 6, tumor necrosis factor alpha, and markers of immune activation in HIV-infected subjects receiving interleukin 2. AIDS Res Hum Retroviruses. 2002 Jun. 10; 18(9):661-5). IC485 is an orally administered, small molecule inhibitor of PDE4. Inhibition of PDE4 leads to an increase in the second messenger, cAMP, within cells. This inhibition may in turn reduce the cell's production of tumor necrosis factor alpha (TNF-alpha) and a variety of other inflammatory mediators. IC485 is being evaluated in patients with chronic obstructive pulmonary disease.

The inhibitor can be a PAF receptor antagonist (such as lexipafant, WEB2086, WEB2170, BN-52021 or PMS-601), a PAF degrading-enzyme such as PAF-acetylhydrolase (PAF-AH), or a molecule that regulates the expression of PAF-AH (such as pioglitazone and other PPAR-gamma inhibitors). Lexipafant has been used improve cognitive dysfunction in HIV-infected people (Schifitto G, Sacktor N, Marder K, McDermott M P, McArthur J C, Kieburtz K, Small S, Epstein L G. Randomized trial of the platelet-activating factor antagonist lexipafant in HIV-associated cognitive impairment. Neurological AIDS Research Consortium. Neurology. 1999 Jul. 22; 53(2):391-6). Lexipafant can be administered at for example 500 mg/day. PMS-601 is a PAF receptor antagonist that inhibits proinflammatory cytokine synthesis and HIV replication (Martin M, et al. 2000). TNF-alpha-mediated neuronal apoptosis can also be blocked by co-incubation with PAF acetylhydrolase (PAF-AH) (Perry S W, et al. 1998). Pioglitazone can inhibit PAF-induced morphological changes through PAF-AH (Sumita C, et al. 2004). Phosphatidylcholines (1-O-alcoxy-2-amino-2-desoxy-phosphocholines and 1-pyrene-labeled analogs) have been synthesized and used to examine interactions with recombinant human PAF-AH (Deigner H P, 1999).

The disclosed method can further comprise administering to the subject a therapeutically effective dose of a compound that enhances CNS uptake. Ritonavir influences levels of coadministered drugs in the CNS, due to effects on the activity of drug transporters located at the BBB (Haas D W, et al. 2003).

The disclosed method can further comprise administering to the subject a therapeutically effective dose of a drug that inhibits the P-glycoprotein drug efflux pump, or multidrug resistance-associated proteins at the blood-brain-barrier (BBB). These include LY-335979 (Choo E F, et al. 2000) and PSC-833 and GF120918 (Pgp blockers) (Polli J W, et al. 1999; Kemper E M, et al. 2003) as well as MK571 (a specific Mrp family inhibitor).

The disclosed method can further comprise administering to the subject a therapeutically effective dose of a microglial deactivator. Minocyclin is a potent microglial deactivator (Wu D C, et al. 2002; Yranheikki J, et al. 1998). Further, minocycline can potently inhibit HIV-1 viral production from microglia (Si Q, et al. 2004). Thus, the microglial deactivator can be minocycline. A typical dosage of minocyclin comprises 200 mg/day. Other microglial deactivators that can be used in the present methods include PDE4 inhibitors.

The disclosed method can further comprise administering to the subject a therapeutically effective dose of an inhibitor of glutamate damage. The inhibitor can be a beta-lactam antibiotic such as for example ceftriaxone, which can have direct effects on glutamate transporter expression. When delivered to animals, the beta-lactam ceftriaxone increases both brain expression of GLT1 that inactivates synaptic glutamate (Rothstein J D, et al. 2005) A typical dosage of cephtriaxone is 50 mg/kg/day. A dose-dependent inhibition of high affinity glutamate uptake sites is observed after addition of exogenous recombinant human TNFα to human fetal astrocytes (PHFAs) (Fine S M, et al. 1996). Thus, the inhibitor of glutamate damage can be a TNFα inhibitor or a microglial deactivator, which can have indirect effects on glutamate transporters.

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, the disclosed anti-retroviral compounds and antioxidants can be administered at published dosages, such as those approved for human use, e.g., in the treatment of HIV-1 infection.

A typical daily dosage of valproate used alone can range from about 0.001 mg/kg to up to 50 mg/kg of body weight or more per day, depending on the factors mentioned above. For example, for human subjects, a typical dose of valproate comprises 250 mg twice daily. Provided herein is a method of treating or preventing neurological disease in a subject in need of such treatment or prevention, comprising administering to the subject a composition comprising valproate at a dosage of about 1 to 20 mg/kg per day, including 1, 2, 3, 4, 5, 6, 7, 8, 9, 10, 11, 12, 13, 14, 15, 20 mg/kg per day.

A typical daily dosage of the disclosed inhibitors of hyperpolarization can range from about 0.001 mg/kg to up to 50 mg/kg of body weight or more per day, depending on the factors mentioned above. In one aspect, the disclosed KATP channel antagonists can be administered at from 0.02 mg/kg to about 30 mg/kg of body weight per day. As non-limiting examples, Tolbutamide can be administered at from about 0.25 to 3 g/day; glibenclamide (glyburide) can be administered at from about 1.25 to 20 mg/day; and meglitinide analog (e.g. Repaglinide, A-4166) can be administered at from about 0.5 to 4 mg/day.

A typical daily dosage of the disclosed inhibitors of the compound that enhances CNS uptake, such as Ritonavir, can range from about 0.001 mg/kg to up to 50 mg/kg, including about 4 to 8 mg/kg of body weight or more per day, depending on the factors mentioned above.

A typical daily dosage of the disclosed inhibitors of the drug that inhibits the P-glycoprotein drug efflux pump, such as LY-335979, GF120918, and MK571, can range from about 0.001 mg/kg to up to 50 mg/kg, including about 2-50 mg/kg, 7 to 21 mg/kg, 2 to 16 mg/kg of body weight or more per day, depending on the factors mentioned above.

In another aspect, the disclosed inhibitors of the ECC (e.g., DPI, rotenone, antimycin, myxothiazole, TTFA, and KCN can be administered at from 0.001 mg/kg to 1 mg/kg of body weight per day. In another aspect, the disclosed protonophore (e.g., FCCP, DNP, CCCP, PCP) can be administered at from 0.001 mg/kg to 1 mg/kg of body weight per day. In one aspect, the disclosed beta-adrenergic agonist CL-316,243 can be administered at 0.01 to up to 1 mg/kg, including 0.1 mg/kg, of body weight or more per day.

In another aspect, the disclosed antioxidants can be administered at from 1 mg/day to 1000 mg/day. As non-limiting examples, N-acetylcysteine (NAC) can be administered at from about 600 mg/day to 800 mg/day; Mito-Coenzyme Q10 (Mito-CoQ) can be administered at from about 300 mg/day to 400 mg/day; Mito-VitaminE (Mito-E) can be administered from about 100 to 1000 mg/day); Coenzyme Q10 can be administered from about 300 mg/day to 400 mg/day; and idebenone can be administered at from about 60 mg/day to 120 mg/day.

A typical daily dosage of the disclosed modulators of adenosine receptor signaling used alone can range from about 0.05 to 5 mg/kg of body weight or more per day, depending on the factors mentioned above. In one aspect, the disclosed A_{2A}R antagonists (e.g. ATL455, KW6002 and ZM241685) can be administered at doses ranging from 0.3 to 3 mg/kg of body weight per day; KW6002 can be administered to humans at doses up to 40 mg/day. In another aspect, the disclosed A_{2A}R agonists (e.g. ATL146e, ATL313 and CGS21680) can be administered at from 0.05 to 50 mg/kg of body weight per day.

Any of the compounds described herein can be the pharmaceutically-acceptable salt thereof. In one aspect, pharmaceutically-acceptable salts are prepared by treating the free acid with an appropriate amount of a pharmaceutically-acceptable base. For example, one or more hydrogen atoms of the SO₃H group can be removed with a base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

For example, the GSK-3 inhibitor of the provided method can be sodium valproate, i.e., the sodium salt of valproic acid. Optimally, the GSK-3 inhibitor of the provided method is lithium valproate, i.e., the lithium salt of valproic acid. Thus, provided herein is a method of treating or preventing neurological disease in a subject in need of such treatment or prevention, comprising administering to the subject a composition comprising lithium valproate.

In another aspect, if the compound possesses a basic group, it can be protonated with an acid such as, for example, HCl or H₂SO₄, to produce the cationic salt. For example, the techniques disclosed in U.S. Pat. No. 5,436,229 for producing the sulfate salts of argininal aldehydes, which is incorporated by reference in its entirety, can be used herein. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

It is contemplated that the pharmaceutically-acceptable salts of the compounds described herein can be used as prodrugs or precursors to the active compound prior to the administration. For example, if the active compound is unstable, it can be prepared as its salt form in order to increase stability in dry form (e.g., powder).

The severity of dementia in persons with HIV-1 associated neurologic disease is strongly correlated with the number of macrophages and microglia within the basal ganglia and frontal lobes (Glass, J. D., et al. 1995). Thus, the activation of microglia and brain macrophages plays a crucial role in the induction of neuronal dysfunction and damage. Thus, the herein disclosed agonists of adenosine receptor signaling can inhibit HAD in a subject in part by inhibiting the recruitment of monocytes to the CNS.

The compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. Thus, the disclosed compositions can be administered intracranially intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The compositions may be administered orally or parenterally (e.g., intravenously, intramuscular injection, by intraperitoneal injection, transdermally, extracorporeally, intracranially, topically or the like, including topical intranasal administration or administration by inhalant. As used herein, “intracranial administration” means the direct delivery of substances to the brain including, for example, intrathecal, intracisternal, intraventricular or trans-sphenoidal delivery via catheter or needle. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffier, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

It must be noted that as used herein and in the appended claims, the singular forms “a”, “an”, and “the” include plural reference unless the context clearly dictates otherwise. Thus, for example, reference to “a molecule” includes a plurality of such molecules, reference to “the molecule” is a reference to one or more molecules and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the appended claims. The following examples are set forth below to illustrate the methods and results according to the present invention. These examples are not intended to be inclusive of all aspects of the present invention, but rather to illustrate representative methods and results. These examples are not intended to exclude equivalents and variations of the present invention which are apparent to one skilled in the art.

### EXAMPLES

### Example 1

### Valproic Acid Adjunctive Therapy for HIV-Associated Cognitive Impairment

### Methods

Twenty-two eligible subjects, six without cognitive impairment and 16 with cognitive impairment, were enrolled and block-randomized within impairment strata to receive 250 mg of VPA or placebo twice daily. Cognitive impairment was defined as performance at least one standard deviation below the mean on two or more neuropsychological tests, or at least two standard deviations below the mean on one neuropsychological test, using normative data previously applied by the Dana cohort (Dana 1996). Subjects were evaluated at 2, 6, and 10 weeks for adverse clinical and laboratory experiences. Neuropsychological evaluations (see Table 3) were performed at screening, week 6, and week 10 along with global assessments of functioning (subject and investigator), the Fatigue Severity Scale, and the Center for Epidemiologic Studies Depression Scale. A neurological examination and CD4+/CD8+ counts were performed at screening and week 10, while plasma HIV viral load was measured at baseline and week 10.

Proton (1H) magnetic resonance spectroscopy (MRS) and diffusion tensor imaging (DTI) were performed at baseline and week 10, using a 1.5 Tesla General Electric Signa MRI scanner (with twinspeed gradients and EXCITE11 software). Single-voxel proton spectra were acquired from three locations in the brain: midline of the frontal lobes; right (or left) mid-frontal centrum semi-ovale; and right (or left) basal ganglia (BG), and relative peak areas of N-acetyl aspartate (NAA), creatine (Cr), choline (Cho), and myo-inositol (MI) were determined. The DTI protocol used to calculate fractional anisotropy and diffusion trace values is reported in Table 5.

Tolerability was assessed based on the proportion of subjects able to complete the 10-week study at the original dose of study medication. Safety measures included the occurrences of adverse events and abnormal results on laboratory tests. Measures of efficacy included changes from baseline in: neuropsychological test scores; the Investigator Clinical Global Impression; MRI indices; functioning; and mood. The study was designed to provide approximately 80% power to detect a 45% difference in tolerability (i.e. 95% versus 50%) between the placebo and VPA groups using a one-sided Fisher's exact test at the 5% level of significance.

Changes from baseline were assessed using paired t-tests. Comparisons between treatment arms were based on unequal-variance two-sample t-tests for continuous variables, and Fisher's Exact Tests for categorical variables. Comparisons between treatment arms were adjusted for baseline values using Analysis of Covariance. Primary statistical analyses were performed according to the intention-to-treat principle, using the last-observation-carried-forward imputation strategy for subjects who prematurely dropped out of the study. Secondary analyses excluded such subjects.

### Results

Despite randomization, the VPA group was slightly more neurologically and functionally impaired at baseline (Table 2), likely due to the small sample size. A higher percentage of patients in the control group were taking HAART than in the VPA group, possibly explaining the higher percentage of detectable plasma viral load in the VPA group (Table 2). Baseline cognitive performance (Table 3) and MRS and DTI indices were comparable in the two groups.

TABLE 2
Demographic and clinical characteristics of all participants at baseline.

| | | Placebo | Valproic Acid |
|---|---|---|---|
| | | (n = 11) | (n = 11) |
| Age | | 46.64 (8.91) | 43.73 (7.59) |
| Male/female | | 8/3 | 9/2 |
| Caucasian/African American | | 3/8 | 7/4 |
| Years of Education | | 12.45 (2.42) | 11.73 (1.62) |
| Years HIV+ | | 10.42 (5.08) | 8.27 (3.76) |
| CD4 Count (mm³) | | 386.70 (230.50) | 482.00 (113.37) |
| Plasma HIV RNA copies/ml | %≦50 | 63.64 | 27.27 |
| | %>50 < 10,000 | 27.27 | 36.36 |
| | %≧10,000 | 9.09 | 36.36 |

| | | |
|---|---|---|
| % on HAART | 81.82 | 63.64 |
| Weight (kg) | 86.00 (29.23) | 76.91 (14.23) |
| CES-D Score | 36.70 (12.09) | 42.55 (13.89) |
| FSS | 3.6 (1.41) | 4.11 (1.63) |
| Karnofsky Score* | 95.45 (8.20) | 83.64 (5.05) |
| Macro-Neurological Exam Score* | 4.10 (3.21) | 9.67 (5.74) |
| Motor UPDRS Score | 2.36 (5.05) | 5.36 (7.39) |

Values are mean (standard deviation) unless otherwise indicated.
*2-tailed p < .05 for between group difference.
FSS: Fatigue Severity Scale (Krupp et al. Arch Neurol 1989; 46: 1121-1123)
UPDRS: Unified Parkinson Disease Rating Scale
CES-D: Center of Epidemiologic Studies-Depression Scale

TABLE 3
Neuropsychological test scores of
impaired participants at baseline.

| | |
|---|---|
| Placebo | Valproic Acid |
| (n = 7) | (n = 9) |

Rey Auditory Verbal Memory*

| | | | | |
|---|---|---|---|---|
| Total | 31.86 | (5.46) | 32.56 | (9.17) |
| Trial 5 | 7.29 | (1.50) | 7.56 | (2.19) |
| Recall after Interference | 9.57 | (9.66) | 6.44 | (3.17) |
| Delayed Recall | 3.71 | (1.70) | 5.11 | (2.76) |
| Correct Recognition | 8.57 | (3.82) | 10.56 | (3.13) |
| Digit Symbol* | 38.43 | (10.97) | 40.11 | (11.76) |

Mean Reaction Time in msec.

| | | | | |
|---|---|---|---|---|
| Choice | 456.86 | (97.69) | 486.63 | (108.11) |
| Sequential | 639.00 | (117.42) | 602.88 | (114.43) |
| Grooved Pegboard in sec. | | | | |
| Dominant Hand | 83.86 | (16.76) | 86.33 | (18.73) |
| Nondominant Hand | 96.00 | (32.86) | 93.67 | (20.39) |
| Timed Gait in sec. | 8.43 | (0.79) | 8.61 | (0.58) |
| Composite Neuro- | −2.26 | (4.32) | −3.83 | (8.17) |
| psychological Z score | | | | |

Values are mean (standard deviation).
*Results shown as number of correct responses

Of the 22 enrolled subjects, one subject was randomized but discontinued study participation prior to receiving drug and was included only in the baseline analyses (Table 2). Two subjects (one on placebo and one on VPA) completed week 6, and did not return for week 10. Nineteen of the 21 patients with longitudinal evaluation completed the 10-week study, 15 had MRS/DTI data.

There were no significant safety laboratory changes from baseline to week 10 in either the placebo or VPA group. One patient on VPA had an increase in liver function tests <2.5 times the upper limit of normal at week 6; this patient did not return for week 10 assessments. Subsequent laboratory tests obtained from the primary care provider demonstrated a normalization of these values. A second patient on VPA reported mild new onset of acid reflux during the trial, however, this isolated adverse experience did not require suspension of study medication. CD4⁺ T lymphocyte cell counts did not change significantly in either group from baseline to 10 weeks. Changes in plasma HIV RNA from baseline to week 10 occurred in three subjects on VPA [33,300 to 316 copies/ml (this subject changed antiretroviral therapy); 100,000 to 93,900 copies/ml; and 32,500 to 24,700 copies/ml] and two subjects in the placebo group (12,100 to 50 copies/ml; and 8,640 copies/ml to 16,500 copies/ml).

Changes in cognitive performance among impaired subjects are reported in Table 4. Between group differences on independent neuropsychological tests and the composite z-score were not statistically significant, however, with the exception of the Mean Reaction Time and trial 5 of the Ray Auditory Verbal Memory, all neuropsychological measures including the summary Z score favored the impaired subjects in the VPA group. Similar results were found when the six unimpaired subjects were included in the analysis. No significant difference was seen in the investigator clinical global impression between the placebo and the VPA group.

TABLE 4
Mean changes from baseline to week 10 in neuropsychological test scores of
impaired participants.

| | Placebo | | Valproic Acid | | Treatment | 95% Confidence | |
|---|---|---|---|---|---|---|---|
| Variable | (n = 6) | | (n = 9) | | Effect | Interval | P-value |
| Rey Auditory | | | | | | | |
| Verbal Memory* | | | | | | | |
| Total | 0.67 | (7.34) | 2.89 | (6.83) | 2.22 | (−6.10, 10.53) | 0.57 |
| Trial 5 | 1.17 | (1.83) | 0.78 | (2.05) | −0.38 | (−2.75, 1.98) | 0.73 |
| Recall after | | | | | | | |
| Interference | −5.00 | (10.37) | −0.22 | (2.91) | 1.57 | (−2.54, 5.68) | 0.42 |
| Delayed Recall | −0.67 | (2.58) | 1.11 | (0.78) | 1.82 | (−0.32, 3.97) | 0.09 |
| Correct Recognition | 0.83 | (5.08) | 0.11 | (4.14) | 1.16 | (−3.10, 5.42) | 0.57 |
| Digit Symbol* | 2.00 | (7.32) | 6.56 | (5.83) | 5.02 | (−2.65, 12.69) | 0.18 |
| Mean Reaction Time | | | | | | | |
| (msec^{†}) | | | | | | | |
| Choice | −24.00 | (135.98) | −43.38 | (107.57) | 5.67 | (−69.24, 80.58) | 0.87 |
| Sequential | −64.67 | (123.47) | −8.63 | (74.79) | 29.08 | (−87.44, 145.60) | 0.59 |
| Grooved Pegboard (sec^{†}) | | | | | | | |
| Dominant Hand | 1.00 | (15.59) | −9.33 | (17.63) | −10.56 | (−29.35, 8.22) | 0.24 |
| Nondominant Hand | −2.50 | (15.81) | −6.44 | (17.57) | −4.53 | (−24.78, 15.71) | 0.63 |
| Timed Gait (sec^{†}) | −0.26 | (0.79) | −0.65 | (1.09) | −0.25 | (−1.45, 0.95) | 0.65 |
| Composite Neuropsychological | 2.01 | (3.90) | 4.56 | (6.22) | 2.31 | (−3.73, 8.34) | 0.41 |
| Z-score* | | | | | | | |

Values are mean (standard deviation).
Treatment Effect is the difference in mean change between the valproic acid group and the placebo group, adjusted for the baseline value of the neuropsychological test in an analysis of covariance model.
For non-timed tests (*), a positive value for treatment effect indicates better performance in the valproic acid group.
For timed tests (^{†}), a negative value for treatment effect indicates better performance in the valproic acid group.

There was a significant NAA/Cr increase in the frontal white matter (FWM) (p=0.006) of cognitively impaired subjects on VPA but there were no significant changes in other MRS or DTI indices (Table 5).

TABLE 5
Changes in mean treatment effects in MRS and DTI indices from baseline to
week 10 in VPA vs. placebo.

| | | All Subjects | | Impaired Subjects | |
|---|---|---|---|---|---|
| | | | 95% | | 95% |
| | | Treatment | Confidence | Treatment | Confidence |
| | | Effect | Interval | Effect | Interval |
| NAA/Cr | Mid-Frontal Gray Matter | 0.48 | (−0.28, 1.24) | 0.95¹ | (−0.07, 1.98) |
| | Centrum Semi-Ovale | 0.46² | (−0.04, 0.96) | 0.83³ | (0.31, 1.35) |
| | Basal Ganglia | −0.14 | (−0.44, 0.16) | −0.17 | (−0.56, 0.21) |
| Cho/Cr | Mid-Frontal Gray Matter | 0.10 | (−0.05, 0.24) | 0.17 | (−0.05, 0.38) |
| | Centrum Semi-Ovale | 0.08 | (−0.10, 0.26) | 0.12 | (−0.14, 0.38) |
| | Basal Ganglia | 0.00 | (−0.05, 0.05) | 0.01 | (−0.06, 0.07) |
| MI/Cr | Mid-Frontal Gray Matter | 0.71 | (−0.32, 1.74) | 1.23 | (−0.28, 2.74) |
| | Centrum Semi-Ovale | 0.07 | (−0.31, 0.45) | 0.18 | (−0.37, 0.74) |
| | Basal Ganglia | 0.06 | (−0.16, 0.27) | 0.26⁴ | (−0.01, 0.53) |
| *FA | Mid-Frontal Gray Matter | 0.03 | (−0.01, 0.06) | 0.00 | (−0.03, 0.03) |
| | Centrum Semi-Ovale | 0.01 | (−0.02, 0.04) | 0.00 | (−0.04, 0.04) |
| | Basal Ganglia | −0.02 | (−0.05, 0.01) | −0.01 | (−0.05, 0.03) |
| *Tra | Mid-Frontal Gray Matter | 0.01 | (−0.09, 0.12) | 0.02 | (−0.13, 0.18) |
| | Centrum Semi-Ovale | 0.01 | (−0.05, 0.08) | 0.03 | (−0.06, 0.12) |
| | Basal Ganglia | 0.07 | (−0.03, 0.17) | 0.04 | (−0.11, 0.18) |

Treatment Effect is the difference in mean change between the Valproic Acid group and the Placebo group, adjusted for the baseline value in an analysis of covariance model.
¹p = 0.06;
²p = 0.07;
³p = 0.006;
⁴p = 0.06
NAA: N-acetyl aspartate;
Cr: Creatine,
Cho: Choline;
MI: Myo-inositol (LCmodel software used for spectroscopic analysis)
FA: Fractional anisotropy;
TR: Trace
*The DTI data were acquired with one T2 weighted image (b = 0) plus diffusion-weighted (b = 1000 s/mm²) images along 21 different diffusion-encoded directions for each slice, using a spin-echo echoplanar imaging sequence (TR/TE = 8000/85 ms).

### REFERENCE LIST

Bara-Jimenez W, Sherzai A, Dimitrova T, Favit A, Bibbiani F, Gillespie M, Morris M J, Mouradian M M, Chase T N. Adenosine A_{2A} receptor antagonist treatment of Parkinson's disease. Neurology. 2003 Aug. 12; 61(3):293-6.
Bertrand, J. A., S. Thieffine, A. Vulpetti, C. Cristiani, B. Valsasina, S. Knapp, H. M. Kalisz, and M. Flocco. 2003. Structural characterization of the GSK-3beta active site using selective and non-selective ATP-mimetic inhibitors. J Mol Biol 333:393-407.
Blanchard, F., E. Kinzie, Y. Wang, L. Duplomb, A. Godard, W. A. Held, B. B. Asch, and H. Baumann. 2002. FR901228, an inhibitor of histone deacetylases, increases the cellular responsiveness to IL-6 type cytokines by enhancing the expression of receptor proteins. Oncogene 21:6264-77.
Bodner, A., A. C. Maroney, J. P. Finn, G. Ghadge, R. Roos, and R. J. Miller. 2002. Mixed lineage kinase 3 mediates gp120IIIB-induced neurotoxicity. J Neurochem 82:1424-34.
Bodner, A., P. T. Toth, and R. J. Miller. 2004. Activation of c-Jun N-terminal kinase mediates gp120IIIB- and nucleoside analogue-induced sensory neuron toxicity. Exp Neurol 188:246-53.
Bouchain, G., and D. Delorme. 2003. Novel hydroxamate and anilide derivatives as potent histone deacetylase inhibitors: synthesis and antiproliferative evaluation. Curr Med Chem 10:2359-72.
Chang L, Ernst T, Leonido-Yee M, et al. Highly active antiretroviral therapy reverses brain metabolite abnormalities in mild HIV dementia. Neurol 1999; 53:782-789.
Chen G, Huang L D, Jiang Y M, Manji H K. The mood-stabilizing agent valproate inhibits the activity of glycogen synthase kinase-3. J Neurochem. 1999 March; 72(3):1327-30.
Choi, C. H., K. H. Sun, C. S. An, J. C. Yoo, K. S. Hahm, I. H. Lee, J. K. Sohng, and Y. C. Kim. 2002. Reversal of P-glycoprotein-mediated multidrug resistance by 5,6,7,3′,4′-pentamethoxyflavone (Sinensetin). Biochem Biophys Res Commun 295:832-40.
Choo E F, Leake B, Wandel C, Inamura H, Wood A J, Wilkinson G R, Kim R B. Pharmacological inhibition of P-glycoprotein transport enhances the distribution of HIV-1 protease inhibitors into brain and testes. Drug Metab Dispos. 2000 June; 28(6):655-60.
Chuang, D. M. 2005. The Antiapoptotic Actions of Mood Stabilizers: Molecular Mechanisms and Therapeutic Potentials. Ann N Y Acad Sci 1053:195-204.
Curtin, M., and K. Glaser. 2003. Histone deacetylase inhibitors: the Abbott experience. Curr Med Chem 10:2373-92.
Cysique, L. A., P. Maruff, and B. J. Brew. 2004. Prevalence and pattern of neuropsychological impairment in human immunodeficiency virus-infected/acquired immunodeficiency syndrome (HIV/AIDS) patients across pre- and post-highly active antiretroviral therapy eras: a combined study of two cohorts. J Neurovirol 10:350-7.
Dana Consortium on Therapy for HIV Dementia and Related Cognitive Disorders. Clinical confirmation of the American Academy of Neurology algorithm for HIV-1 associated cognitive motor disorder. Neurol 1996; 47:1247-1253.
Deigner H P, Kinscherf R, Claus R, Fyrnys B, Blencowe C, Hermetter A. Novel reversible, irreversible and fluorescent inhibitors of platelet-activating factor acetylhydrolase as mechanistic probes. Atherosclerosis. 1999 May; 144(1):79-90.
DiCenzo R, Peterson D, Cruttenden K, Morse G, Gelbard H, Schifitto G. Effects of valproic acid coadministration on plasma efavirenz and lopinavir concentrations in human immunodeficiency virus-infected adults. Antimicrob Agents Chemother 2004; 48:4328-4331.
Dou, H., B. Ellison, J. Bradley, A. Kasiyanov, L. Y. Poluektova, H. Xiong, S. Maggirwar, S. Dewhurst, H. A. Gelbard, and H. E. Gendelman. 2005. Neuroprotective mechanisms of lithium in murine human immunodeficiency virus-1 encephalitis. J Neurosci 25:8375-85.
Dou, H., K. Birusingh, J. Faraci, S. Gorantla, L. Y. Poluektova, S. B. Maggirwar, S. Dewhurst, H. A. Gelbard, and H. E. Gendelman. 2003. Neuroprotective activities of sodium valproate in a murine model of human immunodeficiency virus-1 encephalitis. Neurosci 23:9162-70.
Eickholt, B. J., G. J. Towers, W. J. Ryves, D. Eikel, K. Adley, L. M. Ylinen, N. H. Chadborn, A. J. Harwood, H. Nau, and R. S. Williams. 2005. Effects of valproic acid derivatives on inositol trisphosphate depletion, teratogenicity, glycogen synthase kinase-3beta inhibition, and viral replication: a screening approach for new bipolar disorder drugs derived from the valproic acid core structure. Mol Pharmacol 67:1426-33.
Everall, I. P., C. Bell, M. Mallory, D. Langford, A. Adame, E. Rockestein, and E. Masliah. 2002. Lithium ameliorates HIV-gp120-mediated neurotoxicity. Mol Cell Neurosci 21:493-501.
Fine S M, Angel R A, Perry S W, Epstein L G, Rothstein J D, Dewhurst S, Gelbard H A. Tumor necrosis factor alpha inhibits glutamate uptake by primary human astrocytes. Implications for pathogenesis of HIV-1 dementia. J. Biol. Chem. 1996 Jun. 28; 271(26):15303-6.
Glass J D, Fedor H, Wesselingh S L, McArthur J C. Immunocytochemical quantitation of human immunodeficiency virus in the brain: correlations with dementia. Ann Neurol. 1995 November; 38(5):755-62.
Gottlicher, M., S. Minucci, P. Zhu, O. H. Kramer, A. Schimpf, S. Giavara, J. P. Sleeman, F. Lo Coco, C. Nervi, P. G. Pelicci, and T. Heinzel. 2001. Valproic acid defines a novel class of HDAC inhibitors inducing differentiation of transformed cells. Embo J 20:6969-78.
Gruber, A., M. Bjorkholm, L. Brinch, S. Evensen, B. Gustavsson, M. Hedenus, G. Juliusson, E. Lofvenberg, I. Nesthus, B. Simonsson, M. Sjo, L. Stenke, J. M. Tangen, U. Tidefelt, A. M. Uden, C. Paul, and J. Liliemark. 2003. A phase I/II study of the MDR modulator Valspodar (PSC 833) combined with daunorubicin and cytarabine in patients with relapsed and primary refractory acute myeloid leukemia. Leuk Res 27:323-8.
Guengerich, F. P., J. L. Sorrells, S. Schmitt, J. A. Krauser, P. Aryal, and L. Meijer. 2004. Generation of new protein kinase inhibitors utilizing cytochrome p450 mutant enzymes for indigoid synthesis. J Med Chem 47:3236-41.
Haas D W, Johnson B, Nicotera J, Bailey V L, Harris V L, Bowles F B, Raffanti S, Schranz J, Finn T S, Saah A J, Stone J Effects of ritonavir on indinavir pharmacokinetics in cerebrospinal fluid and plasma Antimicrob Agents Chemother. 2003 July; 47(7):2131-7.
Hashimoto, R., N. Takei, K. Shimazu, L. Christ, B. Lu, and D. M. Chuang. 2002. Lithium induces brain-derived neurotrophic factor and activates TrkB in rodent cortical neurons: an essential step for neuroprotection against glutarnate excitotoxicity. Neuropharmacology 43:1173-9.
Jeong, M. R., R. Hashimoto, V. V. Senatorov, K. Fujimaki, M. Ren, M. S. Lee, and D. M. Chuang. 2003. Valproic acid, a mood stabilizer and anticonvulsant, protects rat cerebral cortical neurons from spontaneous cell death: a role of histone deacetylase inhibition. FEBS Lett 542:74-8.
Keddie et al (1996) In vivo characterisation of ZM 241385, a selective adenosine A_{2A} receptor antagonist. Eur. J. Pharmacol. 301 107.
Kemper E M, van Zandbergen A E, Cleypool C, Mos H A, Boogerd W, Beijnen J H, van Tellingen O. Increased penetration of paclitaxel into the brain by inhibition of P-Glycoprotein. Clin Cancer Res. 2003 July; 9(7):2849-55.
Kitazaki, T., M. Oka, Y. Nakamura, J. Tsurutani, S. Doi, M. Yasunaga, M. Takemura, H. Yabuuchi, H. Soda, and S. Kohno. 2005. Gefitinib, an EGFR tyrosine kinase inhibitor, directly inhibits the function of P-glycoprotein in multidrug resistant cancer cells. Lung Cancer 49:337-43.
Klein P S, Melton D A. A molecular mechanism for the effect of lithium on development. Proc Natl Acad Sci USA. 1996 Aug. 6; 93(16):8455-9.
Klotz K N. Adenosine receptors and their ligands. Naunyn Schmiedebergs Arch Pharmacol. 2000 November; 362(4-5):382-91.
Knockaert, M., K. Wieking, S. Schmitt, M. Leost, K. M. Grant, J. C. Mottram, C. Kunick, and L. Meijer. 2002. Intracellular Targets of Paullones. Identification following affinity purification on immobilized inhibitor. J Biol Chem 277:25493-501.
Kunick, C., K. Lauenroth, M. Leost, L. Meijer, and T. Lemcke. 2004. 1-Azakenpaullone is a selective inhibitor of glycogen synthase kinase-3 beta. Bioorg Med Chem Lett 14:413-6.
Kuo, G. H., C. Prouty, A. DeAngelis, L. Shen, D. J. ONeill, C. Shah, P. J. Connolly, W. V. Murray, B. R. Conway, P. Cheung, L. Westover, J. Z. Xu, R. A. Look, K. T. Demarest, S. Emanuel, S. A. Middleton, L. Jolliffe, M. P. Beavers, and X. Chen. 2003. Synthesis and discovery of macrocyclic polyoxygenated bis-7-azaindolylmaleimides as a novel series of potent and highly selective glycogen synthase kinase-3beta inhibitors. J Med Chem 46:4021-31.
Lappas C M, et al. A2A adenosine receptor induction inhibits IFN-gamma production in murine CD4+ T cells. J. Immunol. 2005 Jan. 15; 174(2):1073-80.
Lehne, G., D. R. Sorensen, G. E. Tjonnfjord, C. Beiske, T. A. Hagve, H. E. Rugstad, and O. P. Clausen. 2002. The cyclosporin PSC 833 increases survival and delays engraftment of human multidrug-resistant leukemia cells in xenotransplanted NOD-SCID mice. Leukemia 16:2388-94.
Lehrman, G., I. B. Hogue, S. Palmer, C. Jennings, C. A. Spina, A. Wiegand, A. L. Landay, R. W. Coombs, D. D. Richman, J. W. Mellors, J. M. Coffin, R. J. Bosch, and D. M. Margolis. 2005. Depletion of latent HIV-1 infection in vivo: a proof-of-concept study. Lancet 366:549-55.
Leost, M., C. Schultz, A. Link, Y. Z. Wu, J. Biernat, E. M. Mandelkow, J. A. Bibb, G. L. Snyder, P. Greengard, D. W. Zaharevitz, R. Gussio, A. M. Senderowicz, E. A. Sausville, C. Kunick, and L. Meijer. 2000. Paullones are potent inhibitors of glycogen synthase kinase-3beta and cyclin-dependent kinase 5/p25. Eur J Biochem 267:5983-94.
Maeda, T., Y. Nagaoka, H. Kuwajima, C. Seno, S. Maruyama, M. Kurotaki, and S. Uesato. 2004. Potent histone deacetylase inhibitors: N-hydroxybenzamides with antitumor activities. Bioorg Med Chem 12:4351-60.
Maeda, Y., M. Nakano, H. Sato, Y. Miyazaki, S. L. Schweiker, J. L. Smith, and A. T. Truesdale. 2004. 4-Acylamino-6-arylfuro[2,3-d]pyrimidines: potent and selective glycogen synthase kinase-3 inhibitors. Bioorg Med Chem Lett 14:3907-11.
Maggirwar, S. B., N. Tong, S. Ramirez, H. A. Gelbard, and S. Dewhurst. 1999. HIV-1 Tat-mediated activation of glycogen synthase kinase-3beta contributes to Tat-mediated neurotoxicity. J Neurochem 73:578-86.
Mai, A., S. Massa, R. Ragno, M. Esposito, G. Sbardella, G. Nocca, R. Scatena, F. Jesacher, P. Loidl, and G. Brosch. 2002. Binding mode analysis of 3-(4-benzoyl-1-methyl-1H-2-pyrrolyl)-N-hydroxy-2-propenamide: a new synthetic histone deacetylase inhibitor inducing histone hyperacetylation, growth inhibition, and terminal cell differentiation. J Med Chem 45:1778-84.
Martin, M., N. Serradji, N. Dereuddre-Bosquet, G. Le Pavec, G. Fichet, A. Lamouri, F. Heymans, J. J. Godfroid, P. Clayette, and D. Dormont. 2000. PMS-601, a new platelet-activating factor receptor antagonist that inhibits human immunodeficiency virus replication and potentiates zidovudine activity in macrophages. Antimicrob Agents Chemother 44:3150-4.
Martinez, A., M. Alonso, A. Castro, C. Perez, and F. J. Moreno. 2002. First non-ATP competitive glycogen synthase kinase 3 beta (GSK-3beta) inhibitors: thiadiazolidinones (TDZD) as potential drugs for the treatment of Alzheimer's disease. Med Chem 45:1292-9.
Meyerhoff D J, MacKay S, Bachman L, et al. Reduced brain N-acetylaspartate suggests neuronal loss in cognitively impaired Human Immunodeficiency Virus-seropositive individuals: In vivo 1H magnetic resonance spectroscopic imaging. Neurol 1993; 43:509-515.
Moog C, Kuntz-Simon G, Caussin-Schwemling C, Obert G. Sodium valproate, an anticonvulsant drug, stimulates human immunodeficiency virus type 1 replication independently of glutathione levels. J Gen Virol 1996; 77:1993-1999.
Mora, A., R. A. Gonzalez-Polo, J. M. Fuentes, G. Soler, and F. Centeno. 1999. Different mechanisms of protection against apoptosis by valproate and Li⁺. Eur J Biochem 266:886-91.
Moule S K, Welsh G I, Edgell N J, Foulstone E J, Proud C G, Denton R M. Regulation of protein kinase B and glycogen synthase kinase-3 by insulin and beta-adrenergic agonists in rat epididymal fat cells. Activation of protein kinase B by wortmannin-sensitive and -insensitive mechanisms. J Biol. Chem. 1997 Mar. 21; 272(12):7713-9.
Nakajima, H., Y. B. Kim, H. Terano, M. Yoshida, and S. Horinouchi. 1998. FR901228, a potent antitumor antibiotic, is a novel histone deacetylase inhibitor. Exp Cell Res 241:126-33.
Nekooeian A A, Tabrizchi R. Effects of CGS 21680, a selective A2A adenosine receptor agonist, on cardiac output and vascular resistance in acute heart failure in the anaesthetized rat. Br J. Pharmacol. 1998 April; 123(8):1666-72.
Neuenburg, J. K., H. R. Brodt, B. G. Herndier, M. Bickel, P. Bacchetti, R. W. Price, R. M. Grant, and W. Schlote. 2002. HIV-related neuropathology, 1985 to 1999: rising prevalence of HIV encephalopathy in the era of highly active antiretroviral therapy. J Acquir Immune Defic Syndr 31:171-7.
Newman, M. J., R. Dixon, and B. Toyonaga. 2002. OC144-093, a novel P glycoprotein inhibitor for the enhancement of anti-epileptic therapy. Novartis Found Symp 243:213-26; discussion 226-30, 231-5.
Nonaka, S., C. J. Hough, and D. M. Chuang. 1998. Chronic lithium treatment robustly protects neurons in the central nervous system against excitotoxicity by inhibiting N-methyl-D-aspartate receptor-mediated calcium influx. Proc Natl Acad Sci USA 95:2642-7.
Olson, D. P., D. T. Scadden, R. T. D'Aquila, and M. P. De Pasquale. 2002. The protease inhibitor ritonavir inhibits the functional activity of the multidrug resistance related-protein I (MRP-1). Aids 16:1743-7.
O'Neill, D. J., L. Shen, C. Prouty, B. R. Conway, L. Westover, J. Z. Xu, H. C. Zhang, B. E. Maryanoff, W. V. Murray, K. T. Demarest, and G. H. Kuo. 2004. Design, synthesis, and biological evaluation of novel 7-azaindolyl-heteroaryl-maleimides as potent and selective glycogen synthase kinase-3beta (GSK-3beta) inhibitors. Bioorg Med Chem 12:3167-85.
Ortega, M. A., M. E. Montoya, B. Zarranz, A. Jaso, I. Aldana, S. Leclerc, L. Meijer, and A. Monge. 2002. Pyrazolo[3,4-b]quinoxalines. A new class of cyclin-dependent kinases inhibitors. Bioorg Med Chem 10:2177-84.
Perry S W, Hamilton J A, Tjoelker L W, Dbaibo G, Dzenko K A, Epstein L G, Hannun Y, Whittaker J S, Dewhurst S, Gelbard H A. Platelet-activating factor receptor activation. An initiator step in HIV-1 neuropathogenesis. J Biol. Chem. 1998 Jul. 10; 273(28):17660-4.
Phiel, C. J., F. Zhang, E. Y. Huang, M. G. Guenther, M. A. Lazar, and P. S. Klein. 2001. Histone deacetylase is a direct target of valproic acid, a potent anticonvulsant, mood stabilizer, and teratogen. J Biol Chem 276:36734-41.
Phillis J W. The selective adenosine A₂ receptor agonist, CGS 21680, is a potent depressant of cerebral cortical neuronal activity. Brain Res. 1990 Feb. 19; 509(2):328-30.
Pierce, A. C., E. ter Haar, H. M. Binch, D. P. Kay, S. R. Patel, and P. Li. 2005. CH.O and CH.N hydrogen bonds in ligand design: a novel quinazolin-4-ylthiazol-2-ylamine protein kinase inhibitor. J Med Chem 48:1278-81.
Polli J W, Jarrett J L, Studenberg S D, Humphreys J E, Dennis S W, Brouwer K R, Woolley J L. Role of P-glycoprotein on the CNS disposition of amprenavir (141W94), an HIV protease inhibitor. Pharm Res. 1999 August; 16(8):1206-12.
Poucher et al (1996) Pharmacodynamics of ZM 241385, a potent A₂ₐ adenosine receptor antagonist, after enteric administration in rat, cat and dog. J. Pharm. Pharmacol. 48 601.
Poucher et al. (1995) The in vitro pharmacology of ZM 241385, a potent, non-xanthine, A₂ₐ selective adenosine receptor antagonist. Br. J. Pharmacol. 115 1096.
Remiszewski, S. W. 2003. The discovery of NVP-LAQ824: from concept to clinic. Curr Med Chem 10:2393-402.
Rochais, C., E. Lescot, V. Lisowski, A. Lepailleur, J. S. Santos, R. Bureau, P. Dallemagne, L. Meijer, and S. Rault. 2004. Synthesis and biological evaluation of thienopyrrolizines, a new family of CDK/GSK-3 inhibitors. J Enzyme Inhib Med Chem 19:585-93.
Rothstein, J. D., S. Patel, M. R. Regan, C. Haenggeli, Y. H. Huang, D. E. Bergles, L. Jin, M. Dykes Hoberg, S. Vidensky, D. S. Chung, S. V. Toan, L. I. Bruijn, Z. Z. Su, P. Gupta, and P. B. Fisher. 2005. Beta-lactam antibiotics offer neuroprotection by increasing glutamate transporter expression. Nature 433:73-7.
Rubin, E. H., D. P. de Alwis, I. Pouliquen, L. Green, P. Marder, Y. Lin, R. Musanti, S. L. Grospe, S. L. Smith, D. L. Toppmeyer, J. Much, M. Kane, A. Chaudhary, C. Jordan, M. Burgess, and C. A. Slapak. 2002. A phase I trial of a potent P-glycoprotein inhibitor, Zosuquidar.3HCl trihydrochloride (LY335979), administered orally in combination with doxorubicin in patients with advanced malignancies. Clin Cancer Res 8:3710-7.
Sacktor, N., M. P. McDermott, K. Marder, G. Schifitto, O. A. Selnes, J. C. McArthur, Y. Stem, S. Albert, D. Palumbo, K. Kieburtz, J. A. De Marcaida, B. Cohen, and L. Epstein. 2002. HIV-associated cognitive impairment before and after the advent of combination therapy. J Neurovirol 8:136-42.
Sadanand, V., J. Kankesan, A. Yusuf, C. Stewart, J. T. Rutka, J. J. Thiessen, V. Ling, P. M. Rao, S. Rajalakshmi, and D. S. Sarma. 2003. Effect of PSC 833, a potent inhibitor of P-glycoprotein, on the growth of astrocytoma cells in vitro. Cancer Lett 198:21-7.
Schifitto, G., N. Sacktor, K. Marder, M. P. McDermott, J. C. McArthur, K. Kieburtz, S. Small, and L. G. Epstein. 1999. Randomized trial of the platelet-activating factor antagonist lexipafant in HIV-associated cognitive impairment. Neurological AIDS Research Consortium. Neurology 53:391-6.
Serradji, N., M. Martin, O. Bensaid, S. Cistemino, C. Rousselle, N. Dereuddre-Bosquet, J. Huet, C. Redeuilh, A. Lamouri, C. Z. Dong, P. Clayette, J. M. Scherrmann, D. Dormont, and F. Heymans. 2004. Structure-activity relationships in platelet-activating factor. 12. Synthesis and biological evaluation of platelet-activating factor antagonists with anti-HIV-1 activity. J Med Chem 47:6410-9.
Serradji, N., O. Bensaid, M. Martin, E. Kan, N. Dereuddre-Bosquet, C. Redeuilh, J. Huet, F. Heymans, A. Lamouri, P. Clayette, C. Z. Dong, D. Dormont, and J. J. Godfroid. 2000. Structure-activity relationships in platelet-activating factor (PAF). 10. From PAF antagonism to inhibition of HIV-1 replication. J Med Chem 43:2149-54.
Shen, L., C. Prouty, B. R. Conway, L. Westover, J. Z. Xu, R. A. Look, X. Chen, M. P. Beavers, J. Roberts, W. V. Murray, K. T. Demarest, and G. H. Kuo. 2004. Synthesis and biological evaluation of novel macrocyclic bis-7-azaindolylmaleimides as potent and highly selective glycogen synthase kinase-3 beta (GSK-3 beta) inhibitors. Bioorg Med Chem 12:1239-55.
Si, Q., M. Cosenza, M. O. Kim, M. L. Zhao, M. Brownlee, H. Goldstein, and S. Lee. 2004. A novel action of minocycline: inhibition of human immunodeficiency virus type 1 infection in microglia. J Neurovirol 10:284-92.
Stankoff B, Tourbah A, Suarez S, et al. Clinical and spectroscopic improvement in HIV associated cognitive impairment. Neurol 2001; 56:112-115.
Sumita C, Maeda M, Fujio Y, Kim J, Fujitsu J, Kasayama S, Yamamoto I, Azuma J. Pioglitazone induces plasma platelet activating factor-acetylhydrolase and inhibits platelet activating factor-mediated cytoskeletal reorganization in macrophage. Biochim Biophys Acta. 2004 Aug. 4; 1673(3):115-21.
Suzuki, T., A. Matsuura, A. Kouketsu, H. Nakagawa, and N. Miyata. 2005. Identification of a potent non-hydroxamate histone deacetylase inhibitor by mechanism-based drug design. Bioorg Med Chem Lett 15:331-5.
Suzuki, T., Y. Nagano, A. Kouketsu, A. Matsuura, S. Maruyama, M. Kurotaki, H. Nakagawa, and N. Miyata. 2005. Novel inhibitors of human histone deacetylases: design, synthesis, enzyme inhibition, and cancer cell growth inhibition of SAHA-based non-hydroxamates. J Med Chem 48:1019-32.
Tong, N., J. F. Sanchez, S. B. Maggirwar, S. H. Ramirez, H. Guo, S. Dewhurst, and H. A. Gelbard. 2001. Activation of glycogen synthase kinase 3 beta (GSK-3beta) by platelet activating factor mediates migration and cell death in cerebellar granule neurons. Eur J Neurosci 13:1913-22.
Tozzi, V., P. Balestra, P. Lorenzini, R. Bellagamba, S. Galgani, A. Corpolongo, C. Vlassi, D. Larussa, M. Zaccarelli, P. Noto, U. Visco-Comandini, M. Giulianelli, G. Ippolito, A. Antinori, and P. Narciso. 2005. Prevalence and risk factors for human immunodeficiency virus-associated neurocognitive impairment, 1996 to 2002: results from an urban observational cohort. J Neurovirol 11:265-73.
Ueda, H., H. Nakajima, Y. Hori, T. Fujita, M. Nishimura, T. Goto, and M. Okuhara. 1994. FR901228, a novel antitumor bicyclic depsipeptide produced by Chromobacterium violaceum No. 968.1. Taxonomy, fermentation, isolation, physico-chemical and biological properties, and antitumor activity. J Antibiot (Tokyo) 47:301-10.
Valcour, V. G., C. M. Shikuma, M. R. Watters, and N. C. Sacktor. 2004. Cognitive impairment in older HIV-1-seropositive individuals: prevalence and potential mechanisms. Aids 18 Suppl 1:S79-86.
Vasdev, N., A. Garcia, W. T. Stableford, A. B. Young, J. H. Meyer, S. Houle, and A. A. Wilson. 2005. Synthesis and ex vivo evaluation of carbon-11 labelled N-(4-methoxybenzyl)-N′-(5-nitro-1,3-thiazol-2-yl)urea ([(11)C]AR-A014418): A radiolabelled glycogen synthase kinase-3beta specific inhibitor for PET studies. Bioorg Med Chem Lett 15:5270-3.
Wang, E., C. N. Casciano, R. P. Clement, and W. W. Johnson. 2001. The farnesyl protein transferase inhibitor SCH66336 is a potent inhibitor of MDR1 product P-glycoprotein. Cancer Res 61:7525-9.
Wang, S., A. Folkes, I. Chuckowree, X. Cockcroft, S. Sohal, W. Miller, J. Milton, S. P. Wren, N. Vicker, P. Depledge, J. Scott, L. Smith, H. Jones, P. Mistry, R. Faint, D. Thompson, and S. Cocks. 2004. Studies on pyrrolopyrimidines as selective inhibitors of multidrug-resistance-associated protein in multidrug resistance. J Med Chem 47:1329-38.
Ward, K. W., and L. M. Azzarano. 2004. Preclinical pharmacokinetic properties of the P-glycoprotein inhibitor GF120918A (HCl salt of GF120918, 9,10-dihydro-5-methoxy-9-oxo-N-[4-[2-(1,2,3,4-tetrahydro-6,7-dimethoxy-2-isoquinolinyl)ethyl]phenyl]-4-acridine-carboxamide) in the mouse, rat, dog, and monkey. J Pharmacol Exp Ther 310:703-9.
Weiss, J., S. M. Dormann, M. Martin-Facklam, C. J. Kerpen, N. Ketabi-Kiyanvash, and W. E. Haefeli. 2003. Inhibition of P-glycoprotein by newer antidepressants. J Pharmacol Exp Ther 305:197-204.
Wittich, S., H. Scherf, C. Xie, G. Brosch, P. Loidl, C. Gerhauser, and M. Jung. 2002. Structure-activity relationships on phenylalanine-containing inhibitors of histone deacetylase: in vitro enzyme inhibition, induction of differentiation, and inhibition of proliferation in Friend leukemic cells. J Med Chem 45:3296-309.
Woo, S. H., S. Frechette, E. Abou Khalil, G. Bouchain, A. Vaisburg, N. Bernstein, O. Moradei, S. Leit, M. Allan, M. Fournel, M. C. Trachy-Bourget, Z. Li, J. M. Besterman, and D. Delorme. 2002. Structurally simple trichostatin A-like straight chain hydroxamates as potent histone deacetylase inhibitors. J Med Chem 45:2877-85.
Wu D C, Jackson-Lewis V, Vila M, Tieu K, Teismann P, Vadseth C, Choi D K, Ischiropoulos H, Przedborski S. Blockade of microglial activation is neuroprotective in the 1-methyl-4-phenyl-1,2,3,6-tetrahydropyridine mouse model of Parkinson disease. J. Neurosci. 2002 Mar. 1; 22(5):1763-71.
Xu, J., J. Culman, A. Blume, S. Brecht, and P. Gohlke. 2003. Chronic treatment with a low dose of lithium protects the brain against ischemic injury by reducing apoptotic death. Stroke 34:1287-92.
Ylisastigui, L., N. M. Archin, G. Lehrman, R. J. Bosch, and D. M. Margolis. 2004. Coaxing HIV-1 from resting CD4 T cells: histone deacetylase inhibition allows latent viral expression. Aids 18:1101-8.
Yijanheikki J, Keinanen R, Pellikka M, Hokfelt T, Koistinaho J. Tetracyclines inhibit microglial activation and are neuroprotective in global brain ischemia. Proc Natl Acad Sci USA. 1998 Dec. 22; 95(26):15769-74.
Yurek-George, A., F. Habens, M. Brimmell, G. Packham, and A. Ganesan. 2004. Total synthesis of spiruchostatin A, a potent histone deacetylase inhibitor. J Am Chem Soc 126:1030-1.
Zhang, X. D., S. K. Gillespie, J. M. Borrow, and P. Hersey. 2004. The histone deacetylase inhibitor suberic bishydroxamate regulates the expression of multiple apoptotic mediators and induces mitochondria-dependent apoptosis of melanoma cells. Mol Cancer Ther 3:425-35.
Zink, M. C., J. Uhrlaub, J. DeWitt, T. Voelker, B. Bullock, J. Mankowski, P. Tarwater, J. Clements, and S. Barber. 2005. Neuroprotective and anti-human immunodeficiency virus activity of minocycline. Jama 293:2003-11.

1. A method of treating or preventing HIV-1 associated dementia (HAD) in a subject in need of such treatment or prevention, comprising administering to the subject a therapeutically effective dose of a GSK-3 inhibitor.

2. The method of claim 1, further comprising diagnosing the subject with HAD.

3. The method of claim 1, wherein the HAD is minor cognitive minor motor disease (MCMD).

4. The method of claim 1, wherein the GSK-3 inhibitor inhibits GSK-3β.

5. The method of claim 4, wherein the GSK-3 inhibitor is a thienopyrrolizine, indigo derivative, indirubin derivative, or paullone.

6. The method of claim 1, wherein the GSK-3 inhibitor is lithium.

7. The method of claim 1, wherein the GSK-3 inhibitor is valproic acid.

8. The method of claim 1, wherein the GSK-3 inhibitor is an analog or derivative of valproic acid.

9-44. (canceled)

45. A method of treating or preventing HIV-1 associated dementia (HAD) in a human subject in need of such treatment or prevention, comprising administering to the subject a therapeutically effective dose of Valproic acid.

46. A method of treating or preventing neurological disease in a subject in need of such treatment or prevention, comprising administering to the subject a therapeutically effective dose of lithium valproate.


SEQUENCE LISTING (1 sequence)

SEQ ID NO: 1 - protein, 10 aa, Artificial Sequence
  Description of Artificial Sequence note = synthetic construct
GRPRTTSFAE